(19) United States
(12) Patent Application Publication
(10) Pub. No.: US 2004/0171085 A1
(43) Pub. Date: Sep. 2, 2004

(54) Methods and compositions for treating neurodegenerative diseases

(75) Inventor: Claudio A.P. Joazeiro, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IRM LLC, Hamilton (BM)

(21) Appl. No.: 10/701,919

(22) Filed: Nov. 4, 2003

Related U.S. Application Data
(60) Provisional application No. 60/423,961, filed on Nov. 4, 2002.

(51) Int. Cl. G01N 33/00; G01N 33/53; G01N 33/567

(52) U.S. Cl. 435/7.2

(57) ABSTRACT

Methods and compositions for treating neurodegenerative diseases by modulating the interaction of ubiquitin to ubiquitin binding motifs are provided.

### CROSS-REFERENCE TO RELATED APPLICATIONS

The present application claims benefit of priority to U.S. Provisional Patent Application No. 60/423,961, filed Nov. 4, 2002, which is incorporated by reference in its entirety for all purposes.

### BACKGROUND OF THE INVENTION

A variety of proteins contain repeats of glutamine residues, encoded by the codon CAG. These repeats are prone to instability and sometimes expansion. That is, the number of contiguous glutamine residues in a protein increase. Expansion of glutamine repeats, also referred to as polyglutamine (polyQ) repeats, in protein often results in formation of protein aggregates referred to as polyQ aggregates. PolyQ aggregate formation is linked to a number of neurodegenerative diseases, including Huntington's disease, spinocerebellar ataxas (SCA) spinobulbar muscular atrophy (Kennedy disease) and dentatorubropallidoluysian atrophy. These diseases are typically inherited and are manifested late in an individual's life.

To date, it has been unclear how polyQ aggregates cause neurodegenerative disease. The present invention addresses these and other problems.

### BRIEF SUMMARY OF THE INVENTION

The present invention provides methods of identifying an agent for treating a neurodegenerative disease. In some embodiments, the method comprises identifying an agent that inhibits binding of ubiquitin to a ubiquitin binding motif of a polypeptide. In some embodiments, the methods comprise selecting an agent that inhibits the formation of poly glutamine aggregates in a cell and determining whether the agent inhibits binding of ubiquitin to a ubiquitin binding motif of a polypeptide. In some embodiments, the polypeptide comprises a reporter gene product.

In some embodiments, the methods further comprise selecting an agent that delays or prevents symptoms of neurodegenerative disease in an animal. In some embodiments, the methods comprise contacting an agent to a polypeptide comprising a ubiquitin binding motif and selecting an agent that binds to the ubiquitin binding motif. In some embodiments, the polypeptide is ataxin-3. In some embodiments, the polypeptide is p62/Sequestosome-1.

In some embodiments, the selecting step comprises administering the agent to an animal. In some embodiments, the identifying step comprises contacting a agent to a polypeptide comprising the ubiquitin binding motif. In some embodiments, the polypeptide is selected from the group consisting of Ataxin-3, Eps15, Hrs, MEKK1, KIAA1578, KIAA0794, Epsins, STAM, S5a, Usp25, HSJ1, p62/Sequestosome-1, Tollip, Ancient Ubiquitous Protein-1, TAK1-binding protein-2 (TAB2), ASC-1 complex subunit p100, autocrine motility factor receptor, HHRAD23A, PLIC-2, KP78, PAR-1B alpha, MARK4, NUB1, and KIAA1860.

In some embodiments, the methods comprise contacting a cell with the agent, wherein the cell expresses a poly-glutamine repeat comprising a first label and the polypeptide comprising a second label; and measuring the ability of the agent to compete with ubiquitin to block recruitment of the polypeptide to the poly glutamine aggregate comprising the poly glutamine repeat. In some embodiments, the first and second labels are fluorescent. In some embodiments, the polypeptide is linked to a solid support.

In some embodiments, the agent is contacted to the polypeptide in the presence of ubiquitin and the binding of ubiquitin to the polypeptide is measured.

The present invention also provides for methods of treating neurodegenerative disease. In some embodiments, the methods comprise administering to a subject in need thereof a therapeutically effective amount of the agent selected by identifying an agent that inhibits binding of ubiquitin to a ubiquitin binding motif of a polypeptide. In some embodiments, the neurodegenerative disease is selected from the group consisting of Parkinson's disease, Alzheimer's disease, Dentatorubro-pallidoluysian Atrophy (DRPLA), Neuronal Intranuclear Hyaline Inclusion Disease (NIHID), dementia with Lewy bodies, multiple system atrophy, Down's Syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Hallervorden-Spatz disease, inclusion body myositis, Jakob-Creutzfeldt disease, Niemann-Pick disease type 3, progressive supranuclear palsy, subacute sclerosing panencephalitis, Spinocerebellar Ataxias, Huntington's disease, Pick's disease, spinal and bulbar muscular atrophy, dentatorubral-pallidoluysian atrophy, and steatohepatitis.

The present invention also provides methods of identifying a gene product that modulates polyglutamine aggregate (polyQ) formation. In some embodiments, the methods comprise expressing a plurality of polynucleotides in a plurality of cells; and detecting the effect of the expression on polyQ aggregate formation in the cells. In some embodiments, the polynucleotides are cDNAs. In some embodiments, the cells are selected from the group consisting of HEK 293 cells and Neuro2a mouse cells. In some embodiments, the cells also express a polypeptide comprising a polyQ sequence. In some embodiments, the polypeptide comprises a reporter gene product; and the aggregates are detected by detecting reporter gene product activity. In some embodiments, the polypeptide is full-length ataxin-3. In some embodiments, the polypeptide is a fragment of ataxin-3.

### Definitions

A “ubiquitin binding motif (UBM)” as used herein refers to a polypeptide sequence that binds to ubiquitin. Examples of ubiquitin binding motifs include the UB-interaction motif described in, e.g., Hofinan & Falquet, Trends. Biochem. Sci. 26:347-350 (2001). Additional UBMs include those described herein (also referred to as PUB-homologous (PUBH) motifs), e.g., (E/D)(D/E)(E/D)X(L/I)XXAφXφSXX(E/D), wherein φ is a hydrophobic residue, X is any amino acid and amino acids in parentheses indicate options at that position with the first amino acid residue most common at that position. A UBM can comprise the motif alone or can be part of a longer polypeptide sequence. Polypeptides comprising ubiquitin binding motifs include, e.g., Ataxin-3, Eps15, Hrs, MEKK1, KIAA1578, KIAA0794, Epsins, STAM, S5a, Usp25, HSJ1 and other proteins of this family (see, e.g., Hofinann and Falquet, Trends Biochem Sci. 26(6):347-350 (2001)) and p62/Sequestosome-1, Tollip, Ancient Ubiquitous Protein-1, TAK1-binding protein-2 (TAB2), ASC-1 complex subunit p100, autocrine motility factor receptor, HHRAD23A, PLIC-2 and related proteins, KP78, PAR-1B alpha, MARK4, NUB1, KIAA1860 and other proteins of the UBA superfamily (see, e.g., Buchberger, Trends Cell Biol. 12(5):216-221 (2002)).

“Polyglutamine repeats” or “polyQ repeats” as used herein refer to a contiguous string of at least five glutamine residues in a polypeptide sequence. The repeats can comprise at least 7, 10, 20, 30 or more glutamine residues.

“PolyQ” aggregates refer to an aggregation of protein in a cell comprising polyQ-containing proteins. PolyQ aggregate accumulation is associated with a number of neurodegenerative diseases. See, e.g., Lunkes, A., and J.-L. Mandel. Nat. Med. 3: 1201-1202 (1997); Davies, S. W., et al. Lancet 351: 131-133 (1998); DiFiglia, M., et al. Science 277: 1990-1993 (1997); Paulson, H. L., et al. Neuron 19: 333-344 (1997); Skinner, P. J., et al. Nature 389: 971-974 (1997).

The term “gene” as used herein refers to a segment of DNA involved in producing a polypeptide chain. “Gene” includes regions preceding and following the coding region (leader and trailer) as well as intervening sequences (introns) between individual coding segments (exons).

“Modulators” are used herein to refer to molecules that inhibit or activate the interaction of ubiquitin (Ub) and a ubiquitin binding motif (UBM) of a polypeptide. Inhibitors or “blockers” are agents that partially or totally block the interaction of ubiquitin and UBMs. Activators are agents that increase the interaction of ubiquitin and UBMs. Modulators include naturally occurring and synthetic ligands, antagonists, agonists, small chemical molecules and the like. Assays for identifying modulators are included herein. Samples or assays comprising a polypeptide of the invention that are treated with a potential modulator are compared to control samples without the modulator to examine the extent of effect. Control samples (not treated with modulators) are assigned a relative activity value of 100%. Inhibition of a Ub-UBM interaction is achieved when the quantity of Ub binding compared to the control is less than about 80%, optionally 50% or 25, 10%, 5% or 1%. Activation of the polypeptide is achieved when the quantity of Ub binding compared to the control is at least 110%, optionally 150%, optionally 200, 300%, 400%, 500%, or 1000-3000% or more higher.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

The terms “identical” or percent “identity,” in the context of two or more nucleic acids or polypeptide sequences, refer to two or more sequences or subsequences that are the same sequences. Two sequences are substantially identical if the two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. The invention provides polypeptides or polynucleotides that are substantially identical to the polypeptides or polynucleotides, respectively, exemplified herein. Optionally, the identity exists over a region that is at least about 50 nucleotides or amino acids in length, or more preferably over a region that is 100 to 500 or 1000 or more nucleotides or amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. MoL Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

### BRIEF DESCRIPTION OF THE DRAWINGS

FIG. 1 illustrates an alignment of PUB-homology (PUBH) motifs/Ubiquitin-Interaction Motifs (UIM) and the domain structure of ataxin-3. FIG. 1a illustrates the alignment of Atx-3 PUBH/UIM motifs. Numbers indicate the position of the first amino acid in human Atx-3. Amino acids conserved in PUBH motifs are indicated at the bottom. φ indicates a hydrophobic amino acid. Lowercase letters indicate less frequently occurring residues that nonetheless preserve a side chain feature. The conservation of an amino acid or of a side chain feature among PUBH motifs is indicated as follows: the darker highlighting indicates a very high conservation (>90%); the lighter highlighting indicates a high conservation (65-90%). FIG. 1b illustrates the domain structure of Atx-3. Numbers indicate amino acid positions.

FIG. 2 illustrates that PUBH motifs, as well as the N-terminal˜170 amino acids, are conserved in Atx-3 orthologs. Alignment of human (Hs), mouse (Mm), chicken (Gg), worm (Ce) and plant (At) Atx-3 are provided. The human isoform MJD1-1 encoding a tract of 22 Q is shown. Note that the Atx-3 polyQ tract is very short in mouse and chicken, and is absent from the worm and plant proteins. Amino acid positions are indicated to the left of each sequence. PUBH motifs are boxed.

### DETAILED DESCRIPTION OF THE INVENTION

I. Introduction

The present invention is based in part on the surprising discovery that recruitment of certain normal cellular proteins into polyQ aggregates is mediated by binding of ubiquitin to ubiquitin binding motifs. As discussed above, formation of polyQ aggregates is linked to development of neurodegenerative disease. The discovery disclosed herein demonstrates that neurodegenerative disease is caused at least in part by the incorporation of normal cellular proteins into polyQ aggregates via ubiquitin binding motifs in the proteins. Since incorporation of proteins into polyQ aggregates is mediated by binding of ubiquitin binding motifs of a protein, neurodegenerative disease can be treated or prevented by blocking the interaction of ubiquitin to ubiquitin binding proteins.

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

II. Purification Of Proteins of the Invention

Either naturally occurring or recombinant polypeptides of the invention can be purified for use in the assays of the invention. Naturally-occurring polypeptides of the invention can be purified from any source. Recombinant polypeptides can be purified from any suitable expression system, including eukaryotic and prokaryotic cells.

The polypeptides of the invention may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et aL, supra).

A number of procedures can be employed when recombinant polypeptides are being purified. For example, proteins having established molecular adhesion properties (e.g., poly-histidine) can be reversibly fused to a polypeptide of the invention. With the appropriate ligand, either protein can be selectively adsorbed to a purification column and then freed from the column in a relatively pure form. The fused protein may be then removed by enzymatic activity. Finally polypeptides can be purified using immunoaffinity columns.

A. Purification of Proteins from Recombinant Bacteria

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook et al., both supra, and will be apparent to those of skill in the art.

The cell suspension is generally centrifuged and the pellet containing the inclusion bodies resuspended in buffer that does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify proteins from bacteria periplasm. Where the protein is exported into the periplasm of the bacteria, the periplasmic fraction of the bacteria can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques For Purifying Proteins

1. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

2. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

Immunoaffinity chromatography using antibodies raised to a variety of affinity tags such as hemagglutinin (HA), FLAG, Xpress, Myc, hexahistidine (His), glutathione S transferase (GST) and the like can be used to purify polypeptides. The His tag will also act as a chelating agent for certain metals (e.g., Ni) and thus the metals can also be used to purify His-containing polypeptides. After purification, the tag is optionally removed by specific proteolytic cleavage.

It will be apparent to one of skill that chromatographic techniques can be performed at any scale and using equipment from many different manufacturers (e.g., Pharmacia Biotech).

III. Identification of Ubiquitin Binding Modulators

Agents (“ubiquitin binding blockers”) that block binding ofubiquitin to ubiquitin binding motifs in proteins are useful for preventing and treating neurodegenerative diseases. The administration to a subject of a therapeutic amount of a ubiquitin binding blocker of the invention is useful to treat a number of neurodegenerative diseases that involve polyQ aggregates, including, e.g., Parkinson's disease, Alzheimer's disease, dementia with Lewy bodies, multiple system atrophy, Down Syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Hallervorden-Spatz disease, inclusion body myositis, Jakob-Creutzfeldt disease, Niemann-Pick disease type 3, progressive supranuclear palsy, subacute sclerosing panencephalitis, Spinocerebellar Ataxias, Huntington's disease, Pick's disease, spinal and bulbar muscular atrophy, dentatorubral-pallidoluysian atrophy, and steatohepatitis.

Preliminary screens to identify potential ubiquitin binding blockers can be conducted by screening for agents capable of binding to ubiquitin binding motifs. Binding assays usually involve contacting a ubiquitin binding motif with one or more test agents and allowing sufficient time for the motif and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound to a polypeptide of the invention or displacement of labeled substrates. The polypeptides of the invention utilized in such assays can be naturally expressed, cloned or synthesized.

In addition, mammalian or yeast two-hybrid approaches (see, e.g., Bartel, P. L. et. al. Methods Enzymol, 254:241 (1995)) can be used to identify polypeptides or other molecules that interact or bind when expressed together in a host cell.

Agents can also be directly selected for their ability to prevent binding of ubiquitin to a ubiquitin binding motif. In this variation, binding assays are performed to detect an agent that interferes with binding between ubiquitin and a ubiquitin binding motif. In some embodiments, a polypeptide comprising the ubiquitin binding motif is labeled and the quantity of label is used to determine the ability of the agent to compete or interfere with the ubiquitin-ubiquitin binding motif interaction.

In some embodiments, cell-based assays are used to detect alterations in polyQ aggregates in cells. Aggregates can be monitored, for example, by expressing polypeptides comprising polyQ and a reporter gene product. Aggregates comprising the reporter gene products can be distinguished (e.g., by fluorescent microscopy) from the otherwise diffuse nature of the signal when aggregates are not formed. The aggregate forms nuclear and peri-nuclear inclusions and can be clearly distinguished from the non-aggregate forming polypeptides which generates diffuse staining throughout the cytoplasm and the nucleus. Exemplary reporter gene products include, e.g., green fluorescent protein (GFP) and luciferase. Exemplary polypeptides comprising polyQ include the full-length ataxin-3 protein as well as fragments of ataxin-3 comprising polyQ. Exemplary cell lines include, e.g., HEK 293 or Neuro2a mouse cell lines

Experiments can be validated using positive controls. For example, the compound MG132 (see, e.g., Kim D, Kim SH, Li GC., Biochem Biophys Res Commun. 254(1):264-8 (1999)), which inhibits proteasome function, can serve as a positive control for increased aggregate formation; co-transfection of cDNA encoding the chaperones Hsp70 and Hsp40 can serve as a positive control for decreased aggregate formation.

In a variation of the above cell-based assays, a plurality of cDNAs (e.g., a cDNA library) is transformed into the cells and then transformed cells, optionally incubated for a period of time (e.g., 24 hours), are then monitored for an effect on aggregate formation. This screening assay can be used to identify cDNAs that modulate any aspect of aggregate formation, including, e.g., ubiquitin binding or, if full-length ataxin-3 is used, processing of the full-length protein to the disease-linked form. These methods are particularly useful for identifying enhancers or suppressors of polyQ aggregation, thereby identifying additional potential drug targets and characterization.

An alternative preliminary screen involves coexpressing a polyQ repeat and a second protein that is incorporated into polyQ aggregates (e.g., a protein comprising a UBM) and screening for agents that prevent recruitment of the second protein into the polyQ aggregate. In some embodiments, a fluorescent label is linked to a polyQ repeat, allowing one to track the cellular location of polyQ aggregates. A second label is used to label the second protein (e.g., ataxin-3, p62 or other protein that are recruited into a polyQ aggregate). The second label can be used to monitor whether the second protein is recruited into aggregates. Co-accumulation of the first and second labels in the same cellular location indicates that the second protein has been recruited into the polyQ aggregate. In some embodiments, the two labels are different fluorescent labels (e.g., green fluorescent protein and cyan fluorescent protein).

A library of compounds can be contacted to the above-described cell to identify agents that prevent recruitment of the second protein into the polyQ aggregate. In some embodiments, the fluorescent labels are monitored using microscopy and/or imaging to identify agents that interfere with recruitment. The screen can be validated by overexpressing an unlabeled ubiquitin binding motif (e.g., amino acids 200-275 of ataxin-3) which competes with the second labeled protein and prevents recruitment of the labeled second protein.

In some embodiments, agents are selected for the ability to inhibit binding of a polypeptide comprising a UBM to ubiquitin. In some embodiments, ubiquitin or the polypeptide are immobilized on a solid support. These embodiments can be particularly useful for high throughput screens. For example, in some embodiments, a polypeptide comprising a UBM is immobilized on a solid support (e.g., a micro titer dish) and incubated with ubiquitin chains in the presence or absence of one or more test compounds. Unbound ubiquitin is washed away and the remaining bound ubiquitin is quantified. Agents that reduce the amount of bound ubiquitin (i.e., that specifically compete with ubiquitin for binding) are then selected for further testing.

The agents tested as ubiquitin binding blockers of the invention can be any small chemical compound, or a biological entity, such as a protein, sugar, nucleic acid or lipid. In some embodiments, nucleic acid libraries (e.g., cDNA libraries) are expressed in transgenic melanopsin knockout animals or their cells or heterologous cells such as HEK 293 or Neuroa2a mouse cells. Alternatively, test compounds will be small organic molecules or peptides. Essentially any chemical compound can be used as a potential modulator or ligand in the assays of the invention, although most often compounds that can be dissolved in aqueous or organic (especially DMSO-based) solutions are used. In some cases, the chemical compounds screened have an average molecular weight of less than 1000 Daltons. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, high throughput screening methods involve providing a combinatorial chemical or peptide library containing a large number of potential therapeutic compounds (potential modulator compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. Nos. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Samples or assays that are treated with a potential blockers (e.g., a “test compound”) are compared to control samples without the test compound, to examine the extent of, e.g., binding to a ubiquitin binding motif or preventing of binding between ubiquitin and a ubiquitin binding motif.. Control samples (untreated with agents) are assigned a relative activity value of 100. Inhibition of binding is achieved when the activity value relative to the control is about 90%, optionally 50%, optionally 25-0%.

Agents that are initially identified by any of the foregoing screening methods can be further tested to validate the apparent activity. Modulators that are selected for further study can be tested for an effect on prevention and treatment of neurodegenerative disease.

For example, the effect of the compound can be assessed in animals. In addition, transgenic animals expressing human polypeptides of the invention can be used to further validate drug candidates. Exemplary animal model systems include those described in, e.g., Janus et al., Physiol Behav. 73(5):873-86 (2001); Lee et al. Neurology. 56(11 Suppl 4):S26-30 (2001); Zoghbi et al., Annu Rev. Neurosci. 23:217-47 (2002); Dawson, Cell 101(2):115-8 (2000); Betarbet, et al. Bioessays 24(4):308-18 (2002)

IV. Administration and Pharmaceutical Compositions

Ubiquitin binding blockers of the invention can be administered directly to the mammalian subject. Administration is by any of the routes normally used for introducing a modulator compound into ultimate contact with the tissue to be treated and is well known to those of skill in the art.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17^{th} ed. 1985)).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, intrathecally or into the eye (e.g., by eye drop or injection). The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to induce a beneficial response in the subject over time, i.e., to prevent or treat a neurodegenerative disease. The optimal dose level for any patient will depend on a variety of factors including the efficacy of the specific modulator employed, the age, body weight, physical activity, and diet of the patient, and on a possible combination with other drug. The size of the dose also will be determined by the existence, nature, and extent of any adverse side-effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side-effects of the modulator at various concentrations, as applied to the mass and overall health of the subject. Administration can be accomplished via single or divided doses.

The modulators of the invention may be used alone or in conjunction with other agents that are known to be beneficial in treating or preventing neurodegenerative disease. The modulators of the invention and an other agent may be coadministered, either in concomitant therapy or in a fixed combination, or they may be administered at separate times.

### EXAMPLE

The following example is offered to illustrate, but not to limit the claimed invention.

### Example 1

This example demonstrates that a ubiquitin binding motif mediates recruitment of proteins into polyQ aggregates.

A hallmark of most neurodegenerative diseases, including those caused by polyglutamine (polyQ) expansion, is the formation of ubiquitin (Ub)-positive protein aggregates in affected neurons. See, e.g., Alves-Rodrigues, et al., Trends Neurosci 21:516-520 (1998); Lowe, et al. Adv. Exp. Med. Biol. 487:169-187 (2001); Giasson, et al. Neuron 31:885-888 (2001). Here we show that Ub-binding motifs in wild-type (wt) Ataxin-3 (Atx-3) and p62/Sequestosome-1, two known factors sequestered into aggregates in several neurodegenerative diseases (Uchihara, T. et al. Acta Neuropathol 102:149-152 (2001); Takahashi, J. et al. J. Neuropathology and Experimental Neurology 60:369-373 (2001); Perez, M. K. et al. J. Cell Biol. 143:1457-1470 (1998); Zatloukal, K. et al. American Journal of Pathology 160:255-263 (2002); Kuusisto, et al. Neuroreports 12:2085-2090 (2001)), are required for the localization of both proteins into polyQ aggregates.

Ub is an 8-kDa protein whose carboxyl-terminus (C-terminus) is covalently attached to lysyl residues of specific substrates (and of Ub itself, to form polymeric chains) via the action of Ub ligases. The best known function of this post-translational modification (ubiquitylation) is to promote substrate recognition by the proteasome degradation machinery. Various 26S proteasome subunits and proteasome-interacting proteins likely recognize poly-ubiquitylated proteins destined for degradation (Finley, D. Nature Cell Biol. 4:E121-E123 (2002)).

The interaction between Atx-3 and Ub chains is mediated by motifs homologous to poly-Ub-binding motifs found in the proteasome subunit Rpn10/S5a. Based on these results and on the finding that the depletion of p62 leads to neuronal dysfunction (Samuels, et al. J. Cell Biochem. 82:452-466 (2001)), we demonstrate that the Ub-mediated recruitment of essential Ub-binding protein(s) into aggregates is a mechanism contributing to neurodegenerative diseases.

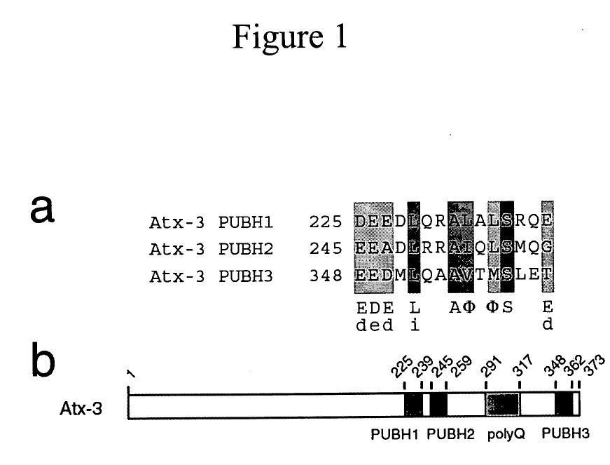

A motif that mediates non-covalent poly-Ub binding (PUB) is conserved between the S. cerevisiae proteasome subunit Rpn10/Mcb1/Sun1 and its homologue, mammalian S5a (Fu, H. et al. J. Biol. Chem. 273:1970-1981 (1998); Young, P., et al. J. Biol. Chem. 273:5461-5467 (1998)). Using database searches, we found PUB-homologous (PUBH) motifs, which are similar to the Ub-Interaction Motif (UIM) (Hofmann, et al. Trends Biochem. Sci. 26:347-350 (2001)), in Ataxin-3 (Atx-3; FIGS. 1a and 1b) and other proteins. Atx-3 also contains a polyQ tract, the expansion of which causes the dominantly inherited Spinocerebellar Ataxia type 3 (SCA-3), or Machado-Joseph's Disease (MJD). Normal Atx-3 is a predominantly cytoplasmic, widely expressed protein of unknown function (Paulson, H. L. et al. Ann. Neurol. 41:453-462 (1997); Zoghbi, H. Y. & Orr, H. T. Annu. Rev. Neurosci. 23:217-247 (2000); Trottier, Y. et al. Neurobiology ofDisease 5:335-347 (199.8)) and the presence of PUBH motifs in Atx-3 raised the possibility that Atx-3 utilizes these motifs to bind to Ub or Ub-like proteins (it was recently shown that the UIM/PUBH motifs found in certain regulators of endocytosis bind to mono-Ub (Polo, S. et al. Nature 416:451-455 (2002); Shih, S.C. et al. Nature Cell Biol. 4:389-393 (2002); Raiborg, C. et al. Nature Cell Biol. 4:394-398 (2002); Bilodeau, P., et al. Nature Cell Biol. 4:534-539 (2002)). To test this possibility, E. coli-purified full-length Atx-3 (1-373) was used in pulldown assays with a mixture of lysine 48 (K48)-linked Ub chains, containing mostly 2-7 Ub. The results show that, like Rpn10/S5a and the 26S proteasome, Atx-3 prefers to bind to chains of four or more Ub. Consistent with this observation, Atx-3 only poorly interacted with mono-Ub.

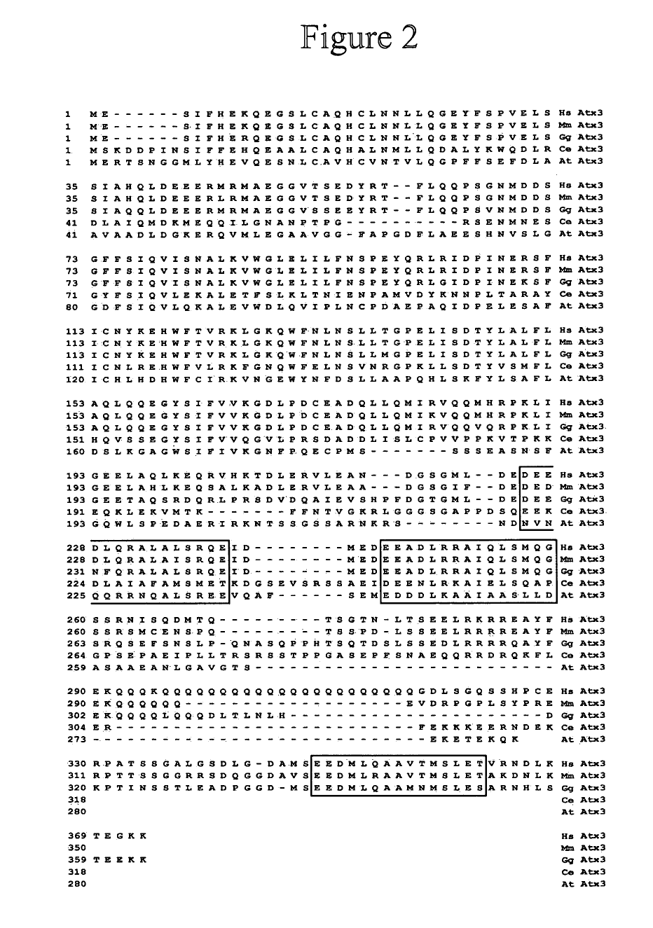

Atx-3 contains three PUBH motifs (FIG. 1a,b), the C-terminal-most one being absent in some splicing variants (e.g., MJD1a (Ichikawa, Y. et al. J. Hum. Genet. 46:413-422 (2001)) and in Atx-3 orthologs (FIG. 2). Deletion of the C-terminal 26 residues, including PUBH3 (Atx-3 1-335), or of the polyQ tract and amino acids C-terminal to it (Atx-3 1-263) was not detrimental for Ub binding. However, further C-terminal truncation leaving only PUBH1 (Atx-3 1-244) or no PUBH motifs (Atx-3 1-222) impaired binding to Ub chains. Conversely, Atx-3 200-275, containing only PUBH1 and PUBH2 and no other recognizable motifs, sufficed for binding to Ub chains.

We investigated whether the interaction between the C-terminal half of Atx-3 and Ub was mediated by PUBH motifs, by replacing a conserved Ser in all three PUBH motifs of Atx-3 (FIG. 1a) with Ala. As PUB and PUBH motifs are predicted to fold as α-helices, these Ser to Ala mutations are not expected to interfere with the motif structure. The triple mutant was defective in binding to Ub. The results presented thus far demonstrate that PUBH/UIM motifs are not dedicated to mono-Ub-binding and the motifs in Atx-3 recognize a surface that is uniquely presented by the Ub tetramer. Atx-3 also interacts with the Ub-like domain of the human homologue of Rad23 (Wang, et al. Hum. Mol. Genet. 9:1795-1803 (2000)), and this interaction is PUBH-dependent.

PUBH motifs also mediate the association of Atx-3 with Ub chains in vivo. FLAG-tagged Atx-3-expressing HEK-293 cells were lysed in NP-40-containing buffer and subjected to immunoprecipitation (IP) with an antibody to the FLAG epitope. Proteins recognized by an antibody against Ub were co-IP'ed along with Atx-3, in a PUBH motif-dependent manner. Mutation of PUBH motifs did not affect Atx-3 protein levels. As expected from the results described herein, the Atx-3 polyQ tract and amino acids C-terminal to it were not required for Ub co-IP, and the conserved N-terminal half of Atx-3 did not co-IP with Ub. The co-IP'ed Ub signal was not due to the covalent attachment of Ub chains to Atx-3 itself, since the Atx-3-Ub co-IP was sensitive to a more stringent, SDS-containing lysis buffer (RIPA). The simplest interpretation of these results is that Atx-3 non-covalently binds to poly-ubiquitylated proteins in vivo through PUBH motif-Ub chain interactions. These interactions are probably critical for the function of Atx-3, since its PUBH motifs are conserved in worm, plant and vertebrate orthologues (FIG. 2).

Expansion of polyQ tracts in Spinocerebellar Ataxias and in Huntington's disease is accompanied by aggregation of the mutant protein and the formation of Ub-rich insoluble inclusions(Zoghbi, H. Y. & Orr, H. T. Annu. Rev. Neurosci. 23:217-247 (2000)). Aggregates in brains of Spinocerebellar Ataxia types 1, 2 and 3, Dentatorubro-pallidoluysian Atrophy (DRPLA), Neuronal Intranuclear Hyaline Inclusion Disease (NIHID) as well as Marinesco Bodies, which are aggregates of unclear relationship to disease, can also recruit the Atx-3 protein encoded by a wild-type (non-expanded) allele and deplete affected neurons of cytoplasmic Atx-3. See, e.g., Uchihara, T. et al. Acta Neuropathol 102:149-152 (2001); Takahashi, J. et al. J. Neuropathology and Experimental Neurology 60:369-373 (2001); Perez, M. K. et al. J. Cell Biol. 143:1457-1470 (1998); Fujigasaki, H. et al. J. Neurol Neurosurg Psychiatry 71:518-520 (2001). It has been argued that such recruitment of normal polyQ tract-containing proteins to polyQ aggregates might occur via polyQ-polyQ interactions. However, in an assay that recapitulates many features that expanded polyQ proteins exhibit in vivo (Perez, M. K. et al. J. Cell Biol. 143:1457-1470 (1998); Paulson, H. L. et al. Neuron 19:333-344 (1997); Zoghbi, H. Y. & Orr, H. T. Annu. Rev. Neurosci. 23:217-247 (2000)), normal Atx-3 was recruited to aggregates formed by a transiently expressed expanded polyQ tract independently of its own polyQ tract.

Our finding that Atx-3 interacts with Ub and the observation that polyQ inclusions, as well as inclusions in other types of neurodegenerative disorders, are Ub-rich prompted the examination of whether the recruitment of wild-type Atx-3 to polyQ aggregates might be mediated by its PUBH motifs. Full-length Atx-3 fused to green fluorescent protein (GFP) and containing or not functional PUBH motifs (wt and S236A/S256A/S359A, respectively) localized mostly to the cytoplasm. For unknown reasons, truncated proteins (1-222, 1-263, 200-275) localized equally in the nucleus and cytoplasm. All proteins expressed at comparable levels. An expanded polyQ tract flanked, at the N- and C-terminal ends, respectively, by 12 and 43 amino acids of the SCA-3-derived Atx-3 isoform MJD1a was fused to cyan-fluorescent protein (CFP-Q78) and formed readily visible aggregates in transfected cells. This disease-associated mutant Atx-3 fragment contains no PUBH motifs. As previously reported, co-expression of an expanded polyQ tract with normal Atx-3 led to recruitment of the latter to aggregates. The results also confirm that the normal length polyQ tract in Atx-3 is not required for the recruitment of Atx-3 into expanded polyQ aggregates, since recruitment can happen with both Atx-3 1-263 and Atx-3 200-275 proteins, which lack the polyQ tract. Furthermore, even upon overexpression, the normal length polyQ tract that is present in Atx-3 S236A/S256A/S359A does not mediate efficient sequestration of this protein in aggregates (the difference in the distribution of Atx-3 S236A/S256A/S359A in cells expressing or not the expanded polyQ tract presumably reflects morphological changes caused by the latter and seems also to depend on Atx-3 sequence). Strikingly, CFP-Q78 aggregates efficiently recruited those Atx-3 proteins bearing functional PUBH motifs, namely, the Atx-3 wt (full-length), Atx-3 1-263 and Atx-3 200-275, but not those without (Atx-3 S236A/S256A/S359A and Atx-3 1-222). Moreover, Atx-3 200-275 contains only two PUBH motifs and no other recognizable motif. Thus, functional PUBH motifs are required and sufficient for recruitment of Atx-3 to polyQ aggregates.

We next investigated whether the recruitment of an unrelated Ub-binding protein to aggregates was similarly mediated by its Ub-binding domain. p62/Sequestosome-1 interacts with Ub via a Ub-Associated (UBA) domain, which is distinct from the PUBH motif. p62 is also present in Ub-rich aggregates formed in neurodegenerative diseases, such as in Alzheimer's, Pick's and Parkinson's (Zatloukal, K. et al. American Journal ofPathology 160:255-263 (2002); Kuusisto, et al. Neuroreports 12:2085-2090 (2001)). Different GFP-p62 fusion proteins were expressed in mammalian cells, and exhibited mostly a punctate cytoplasmic staining pattern. Their co-expression with CFP-Q78 was accompanied by the recruitment to aggregates of GFP-p62 wild-type, but not GFP-p62 lacking amino acids 393-440 (p62 ΔUBA) or GFP-p62 143 I431A, lacking a conserved UBA residue that is important for binding to Ub in the context of human Rad23 (Bertolaet, B. L. et al. Nature Structural Biology 8:417-422 (2001)) (p62 I431 is equivalent to Rad23 L355). Thus, just as Atx-3 requires functional PUBH motifs, p62 requires a functional UBA domain to be recruited to polyQ aggregates.

Protein misfolding due to expansion of a polyQ tract is thought to elicit a cellular response involving chaperones, Ub ligases and the proteasome. Subsequent failure to refold or degrade the expanded polyQ-containing protein is followed by its accumulation in soluble or insoluble aggregates. Insoluble Ub-rich protein aggregates are a hallmark not only of polyQ diseases, but of most neurodegenerative disorders. However, their direct contribution to pathological processes remains controversial. In addition to containing the expanded polyQ protein, Ub, chaperones and the proteasome, aggregates recruit other proteins, including p62 and wt Atx-3. The results presented here indicate that Atx-3 and p62 are recruited to aggregates by a common, Ub-mediated mechanism, and raise the possibility that other Ub-binding proteins are recruited as well. Our data, combined with recent work showing that depletion of p62 via antisense oligonucleotides leads to neuronal dysfunction, suggest a model whereby recruitment of essential Ub-binding proteins into aggregates interferes with cellular function; such interference may be a common mechanism for neurodegenerative disease.

Methods

Pulldown Assays.

Protein expression and purification were done essentially as described (Joazeiro, C. A. et al. Science 286:309-312 (1999)), except that cells were lysed in 50 mM Tris-Cl (pH 8), 500 mM NaCl, 5 mM β-mercaptoethanol, 5 mM imidazole, 10% glycerol and protease inhibitors; Pulldown assays: 200 pmol of His-tagged protein were incubated with 0.5 μg of a mixture of K48-linked Ub chains containing mostly 2-7 Ub units (Ub₂₋₇; Affiniti) for 1.5-4 hours at 4° C. in 1 ml binding buffer [20 mM Tris-Cl (pH 7.5), 100 mM NaCl, 0.1% NP-40, 25 μg/ml BSA], and then with 30 μl of cobalt beads (Talon; Clontech) for 45 min. Proteins pelleted with beads were washed twice in binding buffer without BSA, eluted in SDS sample buffer, subjected to SDS-polyacrylamide gel electrophoresis in 4-20% acrylamide Tris-glycine gel (Invitrogen), and immunoblotted with anti-Ub monoclonal antibody (Zymed).

DNA Constructs.

Atx-3 and p62 cDNA were cloned by PCR from a human brain library (GibcoBRL). We used the Atx-3 isoform MJD1-1 (Ichikawa, Y. et al. J. Hum. Genet. 46:413-422 (2001)) (GenBank NM_{—}004993). Amino acid numbers in this text correspond to that sequence, but with a polyQ tract of 26 residues (which is within the normal range of 13-36 residues). Vectors used were pZsGreen1-C1 (Clontech), pHis8 or pFLAG. Mutations were introduced as follows: in a 50 μl volume, 100 ng of template was combined with 12.5 pmol of each primer, 0.2 mM dNTPs and 2 μl Pfu (Stratagene) in Pfu buffer. After thermocycling (16 cycles of 95° C. for 30 sec, 54° C. for 1 min, 68° C. for 13 min), reactions were incubated with DpnI and transformed into E. coli. When necessary, multiple rounds of mutagenesis were performed (e.g. S236A/S256A/S359A). cDNA encoding truncated proteins was generated either by PCR with internal oligonucleotides or by mutagenesis. Complete coding sequences were verified by sequencing. CFP-Q78 was generated by PCR amplification of an expanded polyQ tract from Atx-3 (isoform MJD1a) using UAS-MJDtr-Q78 (Warrick, J. M. et al. Cell 93:939-949 (1998)) as template and cloning into pCMX-CFP.

Immunoprecipitation.

HEK-293 cells were transfected with FLAG plasmids using FUGENE-6 (Roche). Two days after transfection cells were lysed for 20 min at 4° C. in either 50 mM Tris-Cl pH 7.5, 120 mM NaCl, 1 mM EDTA, 0.1% NP-40 (NP-40 buffer) or 50 mM Tris pH 8.0, 150 mM NaCl, 1% Triton-X100, 0.5% Na-deoxycholate, 0.1% SDS (RIPA buffer) both containing protease inhibitors. Cleared lysates were mixed with beads covalently coupled to the monoclonal FLAG antibody M2 (Sigma) or with uncoupled M2 antibody for 2 h at 4° C. followed by pulldown with protein A-Sepharose beads. Pelleted beads were washed four times with lysis buffer. Bound proteins were eluted with 125 mM Tris-Cl pH 7, 4% SDS and 20% glycerol, separated on SDS-polyacrylamide gels and subjected to immunobloting with polyclonal antibody to Ub (DAKO) or monoclonal antibody to FLAG (M2; Sigma).

PolyQ Aggregate Co-Localization Assay.

HEK293 cells were transfected with FUGENE-6, plated onto poly-L-lysine coated chamber slides (Beckton-Dickinson) the following day, fixed 48 hours later by addition of equal volume of 8% formaldehyde in PBS directly to culture media and mounted in Prolong Antifade media (Molecular Probes). Cells were analyzed by fluorescence microscopy with an Olympus fluorescence microscope and images were captured using an Orca 100 CCD camera (Hamamatsu). Filter sets were Chroma Cyan GFP (excitation 436/20 nm, emission 535/50 nm) and Chroma 41001 (excitation 489/16 nm, emission 480/40 nm). CFP and GFP filter sets were deliberately selected such that excitation wavelengths were narrow and completely non-overlapping.

### Example 2

This example demonstrates screening methods for identifying ubiquitin binding blockers.

Human embryonic kidney (HEK) 293 cells are stably transfected with a DNA construct encoding a cyan-fluorescent protein (CFP)-tagged expanded polyglutamine repeat (CFP-polyQ). Expression of this fusion protein is under control of a regulated promoter. Cells are co-transfected with a construct that allows constitutive expression of green fluorescent protein (GFP)-tagged Ataxin-3 amino acids 200-275. Induction of expression of CFP-polyQ results in the formation of blue aggregates in the nucleus of cells. These aggregates sequester GFP-Ataxin-3, changing its subcellular distribution from the cytoplasm to aggregates. Therefore, aggregates containing Ataxin-3 are both blue and green-fluorescent.

This assay is performed as a microscopy/imaging-based screen for compounds that inhibit the recruitment of Ataxin-3 to aggregates, thereby restricting Ataxin-3 to its cytoplasmic localization despite the formation of aggregates in the same cells. This screen is validated by overexpressing a non-fluorescent fragment of Ataxin-3 containing amino acids 200-275, which competes with GFP-Ataxin-3 for binding to aggregates and prevent recruitment of the latter.

Compounds identified by the above-described screen affect GFP-Ataxin-3 subcellular localization by one of the following mechanisms: (1) by inhibiting aggregation of CFP-polyQ; (2) by inhibiting ubiquitylation of CFP-polyQ aggregates; (3) by inhibiting nuclear transport of GFP-Ataxin-3; and finally, (4) by inhibiting Ataxin-3-polyQ interaction. In order to select for the latter type of compounds, a secondary screen is performed.

Secondary screens for inhibitors of Ataxin-3 (PUBH)-polyQ (ubiquitin) interaction are based on a biochemical assay. His-tagged recombinant Ataxin-3 is immobilized in microtiter plates and is incubated with ubiquitin chains in the presence or absence of library compounds. Unbound ubiquitin chains are washed away and the remaining bound protein will be detected by one of a number of methods, such as ELISA with antibodies specific for ubiquitin, europium-labeled ubiquitin and DELFIA.

Alternatively, a biochemical assay is used as a primary screen and the imaging-based assay is used as a secondary screen.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A method of identifying an agent for treating a neurodegenerative disease, the method comprising,
identifying an agent that inhibits binding of ubiquitin to a ubiquitin binding motif of a polypeptide.

2. The method of claim 1, comprising selecting an agent that inhibits the formation of poly glutamine aggregates in a cell and determining whether the agent inhibits binding of ubiquitin to a ubiquitin binding motif of a polypeptide.

3. The method of claim 2, the polypeptide comprises a reporter gene product.

4. The method of claim 1, further comprising selecting an agent that delays or prevents symptoms of neurodegenerative disease in an animal.

5. The method of claim 1, comprising contacting an agent to a polypeptide comprising a ubiquitin binding motif and selecting an agent that binds to the ubiquitin binding motif.

6. The method of claim 1, wherein the polypeptide is ataxin-3.

7. The method of claim 1, wherein the polypeptide is p62/Sequestosome-1.

8. The method of claim 2, wherein the selecting step comprises administering the agent to an animal.

9. The method of claim 1, wherein the identifying step comprises contacting an agent to a polypeptide comprising the ubiquitin binding motif.

10. The method of claim 1, wherein the polypeptide is selected from the group consisting of Ataxin-3, Eps15, Hrs, MEKK1, KIAA1578, KIAA0794, Epsins, STAM, S5a, Usp25, HSJ1, p62/Sequestosome-1, Tollip, Ancient Ubiquitous Protein-1, TAK1-binding protein-2 (TAB2), ASC-1 complex subunit p100, autocrine motility factor receptor, HHRAD23A, PLIC-2, KP78, PAR-1B alpha, MARK4, NUB1, and KIAA1860.

11. The method of claim 1, comprising contacting a cell with the agent, wherein the cell expresses a poly-glutamine repeat comprising a first label and the polypeptide comprising a second label; and
measuring the ability of the agent to compete with ubiquitin to block recruitment of the polypeptide to the poly glutamine aggregate comprising the poly glutamine repeat.

12. The method of claim 1 1, wherein the first and second labels are fluorescent.

13. The method of claim 1, wherein the polypeptide is linked to a solid support.

14. The method of claim 1, wherein the agent is contacted to the polypeptide in the presence of ubiquitin and the binding of ubiquitin to the polypeptide is measured.

15. A method of treating neurodegenerative disease, the method comprising administering a therapeutically effective amount of the agent selected in claim 1 to a subject in need thereof.

16. The method of claim 15, wherein the neurodegenerative disease is selected from the group consisting of Parkinson's disease, Alzheimer's disease, Dentatorubro-pallidoluysian Atrophy (DRPLA), Neuronal Intranuclear Hyaline Inclusion Disease (NIHID), dementia with Lewy bodies, multiple system atrophy, Down's Syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Hallervorden-Spatz disease, inclusion body myositis, Jakob-Creutzfeldt disease, Niemann-Pick disease type 3, progressive supranuclear palsy, subacute sclerosing panencephalitis, Spinocerebellar Ataxias, Huntington's disease, Pick's disease, spinal and bulbar muscular atrophy, dentatorubral-pallidoluysian atrophy, and steatohepatitis.

17. A method of identifying a gene product that modulates polyglutamine aggregate (polyQ) formation, the method comprising,
expressing a plurality of polynucleotides in a plurality of cells; and
detecting the effect of the expression on polyQ aggregate formation in the cells.

18. The method of claim 17, wherein the polynucleotides are cDNAs.

19. The method of claim 17, wherein the cells are selected from the group consisting of HEK 293 cells and Neuro2a mouse cells.

20. The method of claim 17, wherein the cells also express a polypeptide comprising a polyQ sequence.

21. The method of claim 20, wherein the polypeptide comprises a reporter gene product; and the aggregates are detected by detecting reporter gene product activity.

22. The method of claim 20, wherein the polypeptide is full-length ataxin-3.

23. The method of claim 20, wherein the polypeptide is a fragment of ataxin-3.


SEQUENCE LISTING (12 sequences)

SEQ ID NO: 1 - protein, 15 aa, Artificial Sequence
  Description of Artificial Sequenceataxin-3 (Atx-3) poly-ubiquitin binding (PUB) homology (PUBH) motif/Ubiquitin-Interaction Motif (UIM) PUBH1
DEEDLQRALALSRQE

SEQ ID NO: 2 - protein, 15 aa, Artificial Sequence
  Description of Artificial Sequenceataxin-3 (Atx-3) poly-ubiquitin binding (PUB) homology (PUBH) motif/Ubiquitin-Interaction Motif (UIM) PUBH2
EEADLRRAIQLSMQG

SEQ ID NO: 3 - protein, 15 aa, Artificial Sequence
  Description of Artificial Sequenceataxin-3 (Atx-3) poly-ubiquitin binding (PUB) homology (PUBH) motif/Ubiquitin-Interaction Motif (UIM) PUBH3
EEDMLQAAVTMSLET

SEQ ID NO: 4 - protein, 373 aa, Homo sapiens
  human (Hs) ataxin-3 (Atx-3) isoform MJD1-1
MESIFHEKQEGSLCAQHCLNNLLQGEYFSPVELSSIAHQLDEEERMRMAEGGVTSEDYRTFLQQPSGNMDDSGFFSIQVISNALKVWGLELILFNSPEYQRLRIDPINERSFICNYKEHWFTVRKLGKQWFNLNSLLTGPELISDTYLALFLAQLQQEGYSIFVVKGDLPDCEADQLLQMIRVQQMHRPKLIGEELAQLKEQRVHKTDLERMLEANDGSGMLDEDEEDLQRALALSRQEIDMEDEEADLRRAIQLSMQGSSRNISQDMTQTSGTNLTSEELRKRREAYFEKQQQKQQQQQQQQQQQQQQQQQQQQQQGDLSGQSSHPCERPATSSGALGSDLGDAMSEEDMLQAAVTMSLETVRNDLKTEGKK

SEQ ID NO: 5 - protein, 350 aa, Mus musculus
  mouse (Mm) ataxin-3 (Atx-3) ortholog
MESIFHEKQEGSLCAQHCLNNLLQGEYFSPVELSSIAHQLDEEERLRMAEGGVTSEDYRTFLQQPSGNMDDSGFFSIQVISNALKVWGLELILFNSPEYQRLRIDPINERSFICNYKEHWFTVRKLGKQWFNLNSLLTGPELISDTYLALFLAQLQQEGYSIFVVKGDLPDCEADQLLQMIKVQQMHRPKLIGEELAHLKEQSALKADLERVLEAADGSGIFDEDEDDLQRALAISRQEIDMEDEEADLRRAIQLSMQGSSRSMCENSPQTSSPDLSSEELRRRREAYFEKQQQQQQEVDRPGPLSYPRERPTTSSGGRRSDQGGDAVSEEDMLRAAVTMSLETAKDNLK

SEQ ID NO: 6 - protein, 363 aa, Gallus gallus
  chicken (Gg) ataxin-3 (Atx-3) ortholog
MESIFHERQEGSLCAQHCLNNLLQGEYFSPVELSSIAQQLDEEERMRMAEGGVSSEEYRTFLQQPSVNMDDSGFFSIQVISNALKVWGLELILFNSPEYQRLGIDPINEKSFICNYKEHWFTVRKLGKQWFNLNSLLMGPELISDTYLALFLAQLQQEGYSIFVVKGDLPDCEADQLLQMIRVQQVQRPKLIGEETAQSRDQRLPRSDVDQAIEVSHPFDGTGMLDEDEENFQRALALSRQEIDMEDEEADLRRAIQLSMQGSRQSEFSNSLPQNASQPPHTSQTDSLSSEDLRRRRQAYFEKQQQQLQQQDLTLNLHDKPTINSSTLEADPGGDMSEEDMLQAAMNMSLESARNHLSTEEKK

SEQ ID NO: 7 - protein, 317 aa, Caenorhabditis elegans
  worm (Ce) ataxin-3 (Atx-3) ortholog
MSKDDPINSIFFEHQEAALCAQHALNMLLQDALYKWQDLRDLAIQMDKMEQQILGNANPTPGRSENMNESGYFSIQVLEKALETFSLKLTNIENPAMVDYKNNPLTARAYICNLREHWFVLRKFGNQWFELNSVNRGPKLLSDTYVSMFLHQVSSEGYSIFVVQGVLPRSDADDLISLCPVVPPKVTPKKEQKLEKVMTKFFNTVGKRLGGGSGAPPDSQEEKDLAIAFAMSMETKDGSEVSRSSAEIDEENLRKAIELSQAPGPSEPAEIPLLTRSRSSTPPGASEPFSNAEQQRRDRQKFLERFEKKKEERNDEK

SEQ ID NO: 8 - protein, 280 aa, Arabidopsis thaliana
  plant (At) ataxin-3 (Atx-3) ortholog
MERTSNGGMLYHEVQESNLCAVHCVNTVLQGPFFSEFDLAAVAADLDGKERQVMLEGAAVGGFAPGDFLAEESHNVSLGGDFSIQVLQKALEVWDLQVIPLNCPDAEPAQIDPELESAFICHLHDHWFCIRKVNGEWYNFDSLLAAPQHLSKFYLSAFLDSLKGAGWSIFIVKGNFPQECPMSSSSEASNSFGQWLSPEDAERIRKNTSSGSSARNKRSNDNVNQQRRNQALSREEVQAFSEMEDDDLKAAIAASLLDASAAEANLGAVGTSEKETEKQK

SEQ ID NO: 9 - protein, 5 aa, Artificial Sequence
  Description of Artificial Sequence polyglutamine (polyQ) repeat
QQQQQ

SEQ ID NO: 10 - protein, 30 aa, Artificial Sequence
  Description of Artificial Sequence polyglutamine (polyQ) repeat
QQQQQQQQQQQQQQQQQQQQQQQQQQQQQQ

SEQ ID NO: 11 - protein, 6 aa, Artificial Sequence
  Description of Artificial Sequence hexahistidine (His) affinity tag
HHHHHH

SEQ ID NO: 12 - protein, 26 aa, Artificial Sequence
  Description of Artificial SequencepolyQ tract of 26 residues
QQQQQQQQQQQQQQQQQQQQQQQQQQ